(19) United States
(12) Patent Application Publication
(10) Pub. No.: US 2017/0312191 A1
(43) Pub. Date: Nov. 2, 2017

(54) AGENTS AND METHODS FOR THE TEMPORARY SHAPING OF KERATIN-CONTAINING FIBERS

(71) Applicant: Henkel AG & Co. KGaA, Duesseldorf (DE)

(72) Inventors: Thomas Foerster, Duesseldorf (DE); Rolf Bayersdoerfer, Hamburg (DE); Thorsten Knappe, Schenefeld (DE)

(73) Assignee: Henkel AG & Co. KGaA, Duesseldorf (DE)

(21) Appl. No.: 15/526,056

(22) PCT Filed: Nov. 2, 2015
(86) PCT No.: PCT/EP2015/075379

§ 371 Date: May 11, 2017

(30) Foreign Application Priority Data
Dec. 10, 2014 (DE) 10 2014 225 432.9

(51) Int. Cl. A61K 8/04; B05B 9/00; A61K 8/34; A45D 7/04; A61K 8/92; A61K 8/72; B05B 9/03; A61Q 5/06

(52) U.S. Cl. CPC A61K 8/046; B05B 9/03; B05B 9/002; A61Q 5/06; A45D 7/04; A61K 8/92; A61K 8/72; A61K 8/34; A61K 2800/87

(57) ABSTRACT

The disclosure relates to a cosmetic product, comprising a) a cosmetic preparation, containing, in relation to its total weight, a1) about 45 to about 95 wt. % of at least one polar solvent; a2) about 0.1 to about 25 wt. % of at least one solid material; b) a device for flash evaporation of the cosmetic preparation a), and to a method using corresponding products and to the use of the cosmetic preparation a) as process material in a device for flash evaporation.

### CROSS-REFERENCE TO RELATED APPLICATION

This application is a U.S. National-Stage entry under 35 U.S.C. §371 based on International Application No. PCT/EP2015/075379, filed Nov. 2, 2015, which was published under PCT Article 21(2) and which claims priority to German Application No. 10 2014 225 432.9, filed Dec. 10, 2014, which are all hereby incorporated in their entirety by reference.

### TECHNICAL FIELD

The application relates to the specialist technical field of temporary re-shaping of keratin-containing fibres, in particular human hair. The subject of the application is constituted by specific cosmetic hair formulations which are suitable for application to keratin-containing fibres through a flash evaporation process. In addition, the use of these cosmetic hair formulations in devices for flash evaporation and methods for temporarily re-shaping keratin-containing fibres are the subject of the present application.

### BACKGROUND

A nice-looking hairstyle is nowadays generally regarded as an essential part of a well-groomed appearance. Time and time again, based on current fashion trends, hairstyles that are considered chic are those that can be constructed with many types of hair only using firming active ingredients or that can be maintained for a relatively long period of time up to several days. Hair treatment agents that permanently or temporarily shape the hair therefore play an important role. Whereas, in the case of permanent re-shaping, the chemical structure of the keratin-containing fibres is modified by reduction and oxidation, no such modification of the chemical structure takes place in the case of temporary re-shaping. Corresponding agents for temporary shaping usually contain synthetic polymers and/or waxes as firming active ingredient.

The most important property of an agent for temporarily shaping keratin-containing fibres, also referred to hereinafter as styling agents, lies in providing the treated fibres with the greatest possible hold in the newly modeled form i.e. a form impressed on the fibres. If the keratin-containing fibres are human hair, reference is also made to a strong hairstyle hold or to a high holding power of the styling agent. The hold of a hairstyle is determined fundamentally by the type and quantity of the used firming active ingredients, however the further constituents of the styling agent and also the application form can also have an effect.

In the field of the temporary shaping of keratin-containing fibres, in particular the spray application of corresponding cosmetic preparations is of great importance, wherein the preparations are generally applied as pump sprays or aerosol sprays. For this purpose, the cosmetic preparations are packed in a dispenser from which they are sprayed either by mechanical force or with the aid of a propellant through a valve. Both methods have obvious drawbacks. While pump sprays usually are not suitable for providing a long-lasting uniform spray application of cosmetic hair preparations, aerosol sprays are based on the use of propellants or propellant gases, which on the one hand exert no cosmetic effect and which may otherwise pose a threat to consumers if misused. Against this background there is a need for alternative ways for atomising cosmetic hair preparations. Flash evaporation has proven to be such an alternative spraying method. In this method, which is described for example in international patent application WO 2001/83071 A1 (Henkel), a liquid or pasty, solvent-containing composition in an enclosed space is heated to a temperature which is above the boiling point of the solvent, whereby an overpressure is generated in the composition. When reducing (throttling) the pressure, the liquid evaporates and can then be sprayed for example utilizing a suitable nozzle.

Although the flash evaporation is therefore suitable in principle for the spray application of cosmetic hair preparations, at the same time not all hair cosmetic preparations can be atomised utilizing a flash evaporation process. This is due on the one hand to the need to heat the cosmetic preparation for flash evaporation, and on the other hand to the specifics of the spray mist produced by flash evaporation, for example the droplet size produced and droplet density in the spray mist.

### BRIEF SUMMARY

A cosmetic product is provided herein. The cosmetic product includes a cosmetic preparation, including, based on its total weight, about 45 to about 95 wt % of at least one polar solvent, and about 1.0 to about 25 wt % of at least one fatty substance. The cosmetic product further includes a device for flash evaporation of the cosmetic preparation.

A cosmetic composition is also provided herein. The cosmetic composition includes, based on its total weight, about 45 to about 95 wt % of at least one polar solvent, and about 1.0 to about 25 wt % of at least one fatly substance. The cosmetic composition is utilized as a process material in a device for flash evaporation.

A method for temporarily shaping keratin-containing fibres is also provided herein. The keratin-containing fibres are acted on by a device for flash evaporation with a cosmetic preparation. The cosmetic preparation includes, based on its total weight, about 45 to about 95 wt % of at least one polar solvent, and about 1.0 to about 25 wt % of at least one fatty substance.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide specific cosmetic hair preparations for the temporary shaping of keratin-containing fibres, which preparations, due to their chemical and physical properties, are suitable for the targeted spray application utilizing a device for flash evaporation. Furthermore, the preparations should be suitable to provide a high degree of hold, in particular, a high long-term degree of hold, and a high volume effect after an application utilizing a flash process. It has been shown that, among the large number of known hair-cosmetically effective styling preparations, solvent-containing preparations in particular with a specific proportion of hydrophobic active ingredients are suitable for achieving this object.

A first subject of the present disclosure is thus a cosmetic product, comprising firs
a) a cosmetic preparation, containing, based on its total weight,
   a1) about 45 to about 95 wt % of at least one polar solvent;
   a2) about 1.0 to about 25 wt % of at least one fatty substance;
b) a device for flash evaporation of the cosmetic preparation a).

The cosmetic preparation a) is liquid. Preferred cosmetic preparations are in the form of an emulsion.

Cosmetic preparations as contemplated herein can be provided over a wide viscosity range. Exemplary viscosities are
about 10,000 to about 80,000 mPas, preferably about 20,000 to about 70,000 mPas (20° C., Brookfield Model D220, spindle 5, 5 rpm);
about 5,000 to about 30,000 mPas, preferably about 7,000-20,000 mPas (20° C., Brookfield Model D220, spindle 6, 5 rpm);
about 50,000 to about 300,000 mPas, preferably about 70,000-250,000 mPas (20° C., Brookfield Model D220, TE Helipath. 5 rpm);
about 100,000 to about 1,200,000 mPas, preferably about 150,000-1,000,000 mPas (20° C., Brookfield Model D220, TE, 5 rpm);
about 2,000,000 to about 4,000,000 mPas, preferably about 2,500,000 to about 3,500,000 mPas (20° C., Brookfield Model D220, TE. 1 rpm);
about 1,500,000 to about 4,000,000 mPas, preferably about 2,000,000 to about 3,500,000 mPas (20° C., Brookfield Model D220, TF Helipath, 1 rpm);
about 400,000 to about 2,000,000 mPas, preferably about 500,000 to about 1,500,000 mPas (20° C., Brookfield Model D220, IF, 5 rpm);
about 4,000,000 to about 10,000,000 mPas, preferably about 4,500,000 to about 9,000,000 mPas (20° C., Brookfield Model D220, TF, 1 rpm).

The cosmetic preparation as contemplated herein contains, as first essential constituent, from about 45 to about 95 wt % of at least one polar solvent a1). Preferred cosmetic products are characterised in that the proportion by weight of the polar solvent a1) in the total weight of the cosmetic preparation a) is about 60 to about 92 wt %, preferably about 70 to about 90 wt %. Corresponding agents are characterised by a good cosmetic effect together with good applicability.

To improve the application properties of cosmetic preparations as contemplated herein while minimising the thermal load of any active ingredients or auxiliaries during the flash evaporation process, it has proven advantageous to use polar solvents a1) which have a boiling point (20° C., 1013 mbar) between about 50 and about 110° C., preferably between about 70 and about 105° C. Ethanol, isopropanol, glycerol and water have been found to be particularly suitable here, and are therefore preferred as polar solvents a1).

Particularly preferred polar solvents a1) or solvent systems are characterised in that
the proportion by weight of water and ethanol in the total weight of the polar solvent a1) is preferably at least about 60 wt %, preferably at least about 70 wt %, particularly preferably at least about 80 wt %, and in particular at least about 90 wt %);
the proportion by weight of water in the total weight of the polar solvent a1) is more than about 70 wt %, preferably more than about 80 wt %, and in particular more than about 90 wt %.

A second essential constituent of cosmetic compositions as contemplated herein is the fatty substance a2). In terms of manufacturability, ease of application and cosmetic effect of cosmetic compositions as contemplated herein, it has proven to be advantageous if the proportion by weight of the thickener a2) in the total weight of the cosmetic preparation a) is 2.0 to 22 wt %, preferably 4.0 to 20 wt %.

Hydrophobic substances that are essentially insoluble in water are referred to as “fatty substances”. The solubility of the fatty substances in water (20° C.) is preferably less than about 5 g per 100 g of water, preferably less than about 1 g per 100 g of water and in particular less than about 0.1 g per 100 g water.

The fatty substances may be in solid or liquid form under normal conditions. A first group of preferred fatty substances a2) is formed by the waxes. The wax a2) may be of natural or synthetic origin. Preferred waxes melt above about 40° C., particularly preferably above about 50° C., in particular at temperatures between about 50° C. and about 90° C.

The following can be used as contemplated herein as wax a2): solid paraffins or isoparaffins, vegetable waxes, such as candelilla wax, carnauba wax, esparto grass wax, Japan wax, cork wax, sugar cane wax, ouricury wax, montan wax, sunflower wax, fruit waxes and animal waxes, such as beeswax and other insect waxes, spermaceti, shellac wax, lanolin and uropygial grease, and also mineral waxes, such as ceresin and ozokerite, or the petrochemical waxes, such as petrolatum, paraffin waxes, and microwaxes from polyethylene or polypropylene and polyethylene. It may be advantageous to use hydrogenated or hardened waxes. Furthermore, chemically modified waxes, in particular the hard waxes, for example, montan ester waxes, sasol waxes and hydrogenated jojoba waxes.

Also suitable are the triglycerides of saturated and optionally hydroxylated C16-30 fatly acids, such as hardened triglyceride fats (hydrogenated palm oil, hydrogenated coconut oil, hydrogenated castor oil), glyceryl tribehenate, or glyceryl tri-12-hydroxystearate, furthermore synthetic full esters of fatty acids and glycols (for example Syncrowachs®) or polyols with 2-6 carbon atoms, fatty acid monoalkanolamides with a C12-22 acyl group and C2-4 alkanol group, esters of saturated and/or unsaturated, branched and/or unbranched alkane carboxylic acids having a chain length of 1 to 80 carbon atoms and saturated and/or unsaturated, branched and/or unbranched alcohols having a chain length of about 1 to about 80 carbon atoms, including, for example, synthetic fatty acid fatty alcohol esters such as stearyl stearate or cetyl palmitate, the esters of aromatic carboxylic acids, dicarboxylic acids or hydroxycarboxylic acids (for example 12-hydroxystearic acid) and saturated and/or unsaturated, branched and/or unbranched alcohols having a chain length of about 1 to about 80 carbon atoms, lactides of long-chain hydroxycarboxylic acids and full esters of fatty alcohols, and di- and tricarboxylic acids, for example dicetyl succinate or dicetyl/stearyl adipate, and mixtures of these substances.

The wax components can also be selected from the group of esters of saturated, unbranched alkane carboxylic acids having a chain length of 14 to 44 carbon atoms and saturated, unbranched alcohols having a chain length of 14 to 44 carbon atoms if the wax component or the totality of the wax components is solid at room temperature. The wax components can be selected, for example, from the group of C16-36 alkyl stearates, the C10-40 alkyl stearates, the C20-40 alkylisostearates, the C20-40 dialkyl esters of dimer acids, the C18-38 alkyl hydroxyl stearoyl stearates, the C20-40 alkyl erucates; the C30-50 alkyl beeswax and cetearyl behenate can also be used. Silicone waxes, for example stearyl trimethyl silane/stearyl alcohol, are also potentially advantageous. Preferred wax components are the esters of saturated, monovalent C20-C60 alcohols and saturated C8-C30 monocarboxylic acids, especially a C20-C40 alkyl stearate, preferably obtainable under the name Kesterwachs® K82H from the company Koster Keunen Inc. The wax or the wax components should be solid at about 25° C., but melt in the range of about 35-95° C., with a range of about 45-85° C. being preferred.

Further preferred wax components are fatty alcohols. For example stearyl alcohol, cetyl alcohol, laurel alcohol, myristyl alcohol, arachidyl alcohol, caprylic alcohol, capric alcohol and behenyl alcohol can be used as fatty alcohols.

Preferably, the wax a2) is selected from beeswax (Cera Alba), carnauba wax, candelilla wax, montan wax, cetyl palmitate, microcrystalline waxes (microcrystalline paraffins), and mixtures thereof. The use of a fatty substance a2) selected from the group beeswax (Cera Alba), carnauba wax and microcrystalline waxes (microcrystalline paraffins) is particularly preferred.

Natural, chemically modified and synthetic waxes can be used alone or in combination. The teaching of the disclosure therefore also includes the combined use of several waxes. Furthermore, a number of wax mixtures optionally in admixture with other additives are also commercially available. Under the names “Special Wax 7686 OE” (a mixture of cetyl palmitate, beeswax, microcrystalline wax and polyethylene having a melting range of about 73-75° C., manufacturer: Kahl & Co), Polywax® GP 200 (a mixture of stearyl alcohol and polyethylene glycol stearate with a melting point of about 47-51° C.; manufacturer: Croda) and “Weichceresin® FL 400” (a petrolatum/vaseline oil/wax mixture with a melting point of about 50-54° C.; manufacturer: Parafluid mineral oil company) are examples of mixture that are used with preference as contemplated herein. Another particularly preferred mixture of waxes a2) comprises beeswax and carnauba wax, optionally in combination with microcrystalline wax.

A second group of fatty substances a2) that are preferred as contemplated herein are the oils. The oils a2) may be of natural or synthetic origin. Preferred oils melt below about 10° C., particularly preferably below 0° C.

Preferred cosmetic preparations as contemplated herein contain a) at least one oil selected from the group of silicone oils. The group of silicone oils includes, in particular, the dimethicones, which also include the cyclomethicones, the amino-functional silicones and the dimethiconols. The dimethicones may be both linear and branched and also cyclic, or cyclic and branched. Suitable silicone oils or silicone rubbers are particularly dialkyl and alkylaryl siloxanes, such as dimethyl polysiloxane and methylphenyl polysiloxane, and also the alkoxylated, quaternised or also anionic derivatives thereof.

Further preferred cosmetic preparations a) contain at least one oil from the group of ester oils, i.e. esters of C6-C30 fatty acids with C2-C30 fatty alcohols, preferably monoesters of fatty acids with alcohols having 2 to 24 carbon atoms, such as isopropyl myristate (Rilanit® IPM), isononanoic acid C16-18 alkyl ester (Cetiol SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP) oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) oleic acid decyl ester (Cetiol® V).

The group of fatty substances a2) further comprises the following preferred substances:
liquid paraffin oils, isoparaffin oils and synthetic hydrocarbons and di-n-alkyl ethers containing a total of 12 to 36 carbon atoms, particularly 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecylether and di-tert-butyl ether, di-iso-pentyl ether, di-3-ethyldecyl ether, tent-butyl-n-octyl ether, isopentyl-n-octyl ether and 2-methyl-pentyl-n-octyl ether.
natural (vegetable) oils such as triglycerides and mixtures of triglycerides can be used. Preferred natural oils are soybean oil, rapeseed oil, orange oil, coconut oil, (sweet) almond oil, walnut oil, peach kernel oil, apricot kernel oil, avocado oil, tea tree oil, sesame oil, sunflower oil, tsubaki oil, evening primrose oil, rice bran oil, palm kernel oil, mango oil, meadowfoam seed oil, safflower oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and shea butter.
Dicarboxylic acid esters such as di-n-butyl adipate, di-(2-ethylhexyl) adipate, di-(2-ethylhexyl) succinate and di-isotridecyl acelaat and diol esters, such as ethylene glycol dioleate, ethylene glycol di-isotridecanoate, propylene glycol di-(2-ethylhexanoate), propylene glycol diisostearate, propylene glycol di-pelargonate, butanediol diisostearate, neopentyl glycol caprylate,
symmetrical, asymmetrical or cyclic esters of carbonic acid with fatty alcohols, for example described in DE-OS 197 56 454, glycerol carbonate or dicaprylyl carbonate (Cetiol® CC),
Trifatty acid esters of saturated and/or unsaturated linear and/or branched fatty acids with glycerol,
polyol fatty acid esters, such as the trade product Hydagen® HSP (Cognis) or Sovermol types (Cognis), of fatty acid and/or fatty acid derivatives and polyols having at least two hydroxy groups, and having a carbon chain of 2 to 30 carbon atoms, such as fatty acid esters of trimethylolpropane.

The use of a fatty substance a2) from the group of silicone oils, paraffin oils, vegetable oils and ester oils, preferably from the group of silicone oils, paraffin oils and the vegetable oils, is particularly preferred.

The cosmetic products as contemplated herein comprise, in addition to the cosmetic preparation a), also a device for flash evaporation. The term “flash evaporation” in the context of the present application means the generation of steam with a reduction in the pressure filled in a closed space that is filled with liquid and that is at overpressure (with respect to the surroundings). A corresponding overpressure can be produced for example by heating an amount of the cosmetic preparation a) in an enclosed space to a temperature T1. In the closed space, the liquid at a given temperature T1 has a saturation pressure p1. If the closed space is opened, for example utilizing a valve, to relaxation space that is not at overpressure with the pressure p0<p1, the pressure thus drops in the previously closed space and the cosmetic preparation a), or the solvent or parts of this solvent contained in the cosmetic preparation, vaporises within the spread of the new pressure level. The resulting vapour or spray mist may be used for the application of specific cosmetic preparations.

If the cosmetic preparation a) thus starting from standard conditions (T0=25° C., p0=1,000 bar) is heated in a closed space, the result in addition to a further elevated temperature is an increased pressure of the cosmetic preparation a). This increased pressure can be reduced in a relaxation space to a pressure p0, for example the surrounding air pressure (p0=1,000 bar), whereby at least partial evaporation of the cosmetic preparation a) is achieved.

The cosmetic composition a) can be relieved of pressure directly in the space in which it was previously heated. The heated cosmetic preparation a) at overpressure alternatively can also be transported after heating into a second compartment, in which the pressure is then relieved subsequently.

Flash evaporation is in other words a method in which the cosmetic preparation a) is heated in a closed container utilizing a heater to temperatures above the ambient temperature, wherein in the container a pressure above ambient pressure prevails, and the heated cosmetic preparation a) under increased pressure is then pressure-relieved from the container into the surrounding environment.

A device for flash evaporation is therefore a device which comprises a container and a heater and is designed such that a cosmetic composition a) may be heated in the closed container utilizing the heater to temperatures above the ambient temperature in such a manner that in the container a pressure above ambient pressure is created and the heated cosmetic preparation a) under increased pressure can be pressure-relieved from the container into the surrounding environment.

Simultaneously with or after the pressure relief, the cosmetic preparation a) can be fed to a nozzle, utilizing which, for example, properties of the vapour or spray mist generated by the flash evaporation, in particular the droplet size or the droplet density, but also the spray width and the shape of the spray cone, can be influenced. The use of nozzles, preferably atomising nozzles, is therefore, preferred. The specific type of nozzle or the specific nozzle configuration is defined deliberately as a function of the respective spray characteristics.

In summary, a preferred device for flash evaporation has
b1) a container b1) to be closed and to be opened utilizing a valve, which container defines the closed interior, in which the cosmetic composition can be received,
b2) a heater b2), which makes it possible to heat a cosmetic preparation disposed in the container b1).

The use of an additional nozzle b3) which enables atomisation of the cosmetic preparation a) escaping from the container is particularly preferred. Alternatively to a valve, a closure element acting in a comparable manner and which can close or release a corresponding opening in the container by a corresponding change in position can also be used.

A preferred subject of the present disclosure is a cosmetic product, comprising
a) a cosmetic preparation, containing, based on its total weight,
   a1) about 45 to about 95 wt % of at least one polar solvent;
   a2) about 1.0 to about 25 wt % of at least one fatty substance;
b) a device for flash evaporation of the cosmetic preparation a), wherein the device for flash evaporation comprises a container b1) and a heater h2) and is designed such that
   the cosmetic preparation a) can be received in the interior of the container b1),
   the interior of the container b1) filled at least in part with the cosmetic preparation a) can be closed,
   the cosmetic preparation a) may be heated in the closed interior of the container b1) utilizing the heater b2) with a resultant increase in pressure.

A particularly preferred subject of the present disclosure is therefore a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a), comprising
   b1) a container b1) to be closed and to be opened utilizing a valve,
   b2) a heater, which makes it possible to heat a cosmetic preparation disposed in the container b1),
   b3) a nozzle b3), which enables atomisation of the cosmetic preparation a).

A particularly preferred subject of the present disclosure is in other words a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a), wherein the device for flash evaporation comprises a container b1) and a heater h2 and is designed such that
   the cosmetic preparation a) can be received in the interior of the container b1),
   the interior of the container b1) filled at least in part with the cosmetic preparation a) can be closed,
   the cosmetic preparation a) may be heated in the closed interior of the container b1) utilizing the heater b2) with a resultant increase in pressure,
   the heated cosmetic preparation a) can be pressure-relieved into the surrounding environment from the interior of the container b1) with a reduction in pressure.

The container b1) in Which the cosmetic preparation is heated is designed in a way that makes it possible to fully close off this container with respect to the surrounding environment during the heating of the cosmetic preparation a) and to open this container after the heating so as to allow flash evaporation of the cosmetic preparation a). This can be ensured for example by a component for flow control, in particular a valve.

The container b1) in which the cosmetic preparation is heated is preferably in contact with another container from which the amount of the cosmetic preparation provided for the flash evaporation is transferred prior to heating into the container b1). The access between this storage tank and container b1) is opened and closed here via an appropriate device, such as a valve. This further container is preferably designed in the form of a storage tank, that is to say it preferably comprises a multiple of, for example more than ten times, preferably more than fifty times, the amount of the cosmetic preparation necessary for an evaporation process. In other words, the further container/storage tank preferably has a multiple of, for example, more than ten times the volume, preferably more than twenty times, and in particular more than fifty times the volume of the container b1).

A further particularly preferred subject of the present disclosure is therefore a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a), comprising
   b1) a container b1) to be closed and to be opened utilizing a valve,
   b2) a heater, which makes it possible to heat a cosmetic preparation disposed in the closed container b1),
   b3) a nozzle b3), which enables atomisation of the cosmetic preparation a),
c) a storage tank for the cosmetic preparation a), from which the cosmetic preparation a) can pass into the container b1), wherein
   the access between the storage tank and container b 1) has a component for flow control, which the flow of the cosmetic preparation a) from the storage tank into the container b1) may be interrupted;
   the storage tank has at least ten times the volume, preferably at least twenty times, and in particular at least fifty times the volume of the container b1).

The storage tank is not a pressure container and the cosmetic composition disposed in the storage tank is not under pressure, in other words the pressure inside the storage tank corresponds to the ambient pressure (also air pressure or atmospheric pressure). Appropriate cosmetic products, for example, therefore do not comprise any propellant. Also, the cosmetic product has no pumping device that is capable of releasing or spraying the cosmetic preparation into the surrounding environment without the action of the device for flash evaporation.

A particularly preferred subject of the present disclosure therefore is therefore a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a), comprising
   b1) a container b1) to be closed and to be opened utilizing a valve,
   b2) a heater, which makes it possible to heat a cosmetic preparation disposed in the closed container b1),
   b3) a nozzle b3), which enables atomisation of the cosmetic preparation a),
c) a storage tank for the cosmetic preparation a), from which the cosmetic preparation a) can pass into the container b1), wherein
   the access between the storage tank and container b1) has a component for flow control, which the flow of the cosmetic preparation a) from the storage tank into the container b l) may be interrupted;
   the storage tank has at least ten times the volume, preferably at least fifty the volume of the container b1);
   the pressure inside the storage tank corresponds to the ambient pressure.

A very particularly preferred subject of the present disclosure is therefore a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a) comprising
   b1) a container b1) to be closed and to be opened utilizing a valve,
   b2) a heater, which makes it possible to heat a cosmetic preparation disposed in the closed container b1),
   b3) a nozzle b3), which enables atomisation of the cosmetic preparation a),
c) a storage tank for the cosmetic preparation a), from which the cosmetic preparation a) can pass into the container b1), wherein
   the access between the storage tank and container b1) has a component for flow control, which the flow of the cosmetic preparation a) from the storage tank into the container b1) may be interrupted;
   the storage tank has at least ten times the volume, preferably at least fifty times the volume of the container b1);
   the pressure inside the storage tank corresponds to the ambient pressure and the cosmetic product does not comprise any propellant.

In addition, preferred cosmetic products are those comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a), comprising
   b1) a container b1) to be closed and to be opened utilizing a valve,
   b2) a heater, which makes it possible to heat a cosmetic preparation disposed in the closed container b1),
   b3) a nozzle b3), which enables atomisation of the cosmetic preparation a),
c) a storage tank for the cosmetic preparation a), from which the cosmetic preparation a can pass into the container b1), wherein
   the access between the storage tank and container b1) has a component for flow control, which the flow of the cosmetic preparation a) from the storage tank into the container b1) may be interrupted;
   the storage tank has at least ten times the volume, preferably at least fifty times the volume of the container b1);
   the pressure inside the storage tank corresponds to the ambient pressure,
      wherein the cosmetic product does not have a pumping device which is suitable for releasing or spraying the cosmetic preparation a) without the action of the device for flash evaporation.

In summary, a particularly preferred subject of the present disclosure is therefore a cosmetic product, comprising
a) a cosmetic preparation, containing, based on its total weight
   a1) about 45 to about 95 wt % of at least one polar solvent;
   a2) about 1.0 to about 25 wt % of at least one fatty substance;
b) a device for flash evaporation of the cosmetic preparation a) comprising
   b1) a container b1) to be closed and to be opened utilizing a valve,
   b2) a heater, which makes it possible to heat a cosmetic preparation disposed in the closed container b1),
   b3) a nozzle b3), which enables atomisation of the cosmetic preparation a),
c) a storage tank for the cosmetic preparation a), from which the cosmetic preparation a) can pass into the container b1), wherein
   the access between the storage tank and container b1) has a component for flow control, which the flow of the cosmetic preparation a) from the storage tank into the container b1) may be interrupted;
   the storage tank has at least ten times the volume, preferably at least fifty times the volume of the container b1);
   the pressure inside the storage tank corresponds to the ambient pressure and the product does not comprise any propellant,
      wherein the cosmetic product does not have a pumping device which is suitable for releasing or spraying the cosmetic preparation a) without the action of the device for flash evaporation.

Besides the two above-described constituents a1) and a2), the cosmetic preparations as contemplated herein a) may contain other active ingredients or auxiliaries, wherein in particular active ingredients or auxiliaries that are preferred are those which improve the manufacturability, ease of application and/or cosmetic effect of cosmetic preparations as contemplated herein.

A first example of a preferred further active ingredient and auxiliary are the film-forming polymers a3), of which the use in the cosmetic preparations as contemplated herein a) is particularly preferred.

Permanently and temporarily cationic, anionic, non-ionic or amphoteric polymers are suitable as film-forming polymers a3). These film-forming polymers may be synthetic or of natural origin. Preferred cosmetic preparations a) contain, based on their total weight, about 0.05 to about 20 wt %, preferably about 0.05 to about 15 wt %, and in particular about 0.1 to about 10 wt % of a film-forming polymer a3). This polymer a4) is different from the polymer a2).

Examples of customary film-forming polymers a3) are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Laurel Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Poly acrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

A first group of particularly preferred polymers a3) are the vinylpyrrolidone homopolymers or copolymers. Polymers used with particular preference are:
Polyvinylpyrrolidones (INCI name PVP; CAS number 9003-39-8), such as those sold under the name Luviskol® (BASF);
Copolymers of vinylpyrrolidone and vinyl acetate (INCI name VP/VA Copolymer, CAS number: 25086-89-9), as sold for example under the name Luviskol® VA (BASF);
Copolymers of vinylpyrrolidone and dimethylaminopropyl methacrylamide (INCI name: VP/DMAPA Acrylates Copolymer; CAS number: 175893-71-7), as sold for example under the name Styleze® CC-10 (Ashland);
Copolymers of vinylpyrrolidone with vinyl caprolactam and dimethylaminoethyl methacrylate (INCI name: Vinyl Caprolactam/VP/dimethylaminoethyl Methacrylate Copolymer, CAS number: 102972-64-5), as sold for example under the name Advantage® LC (Ashland).

Because of their cosmetic effect in combination with the fatty substances a2), film-forming polymers that are used with preference as contemplated herein are in particular selected from the group of vinylpyrrolidione homo- and copolymers, preferably from the group of the polyvinylpyrrolidones, the copolymers of vinylpyrrolidone and vinyl acetate, the copolymers of vinylpyrrolidone and dimethylaminopropyl methacrylamide, and the copolymers of vinylpyrrolidone with vinyl caprolactam and dimethylaminoethyl methacrylate.

A second group of particularly preferred polymers a3) are the copolymers of i) N-tert-octylacrylamide, acrylic acid, iii) tert-butylaminoethyl methacrylate iv) and optionally further monomers.

Preferred copolymers a2) preferably consist of at least 90 wt %, preferably at least 95 wt %, and in particular at least about 97 wt % of the monomers N-tert-octyl acrylamide, acrylic acid and tert-butylaminoethyl methacrylate. Particularly preferred copolymers a2) were obtained exclusively from the monomers N-tert-octyl acrylamide, acrylic acid and tert-butylaminoethyl methacrylate.

Particularly preferred are copolymers a21) from the monomers i) N-tert-octylacrylamide, ii) acrylic acid, iii) tert-butylaminoethyl methacrylate, iv) methyl methacrylate, and v) hydroxypropyl methacrylate.

The copolymers a2) described above are sold, for example, under the name Amphomer® (INCI name: Octylacrylamide/Acrylates/Butylaminoethyl Methacrylate Copolymer; CAS number 70801-07-9) by the company National Starch.

A second example of a preferred further active ingredient and auxiliary are the thickeners a4). Preferred thickeners are selected from the group of polymeric organic thickeners. The polymeric organic thickeners may be crosslinked or uncrosslinked.

In terms of manufacturability, ease of application and cosmetic effect of cosmetic compositions as contemplated herein it has proven to be advantageous if the proportion by weight of the thickener a4) in the total weight of the cosmetic preparation a) is about 0.05 to about 10 wt %, preferably about 0.05 to about 7.0 and in particular about 0.1 to about 5.0 wt %.

Examples of customary thickeners a4) are polymeric thickeners having the INCI names Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Saliva (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/Isophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylonitrogens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxystearamide MEA, Isobutylene/Sodium Maleate Copolymer, Lithium Magnesium Silicate, Lithium Magnesium Sodium Silicate, Macrocystis Pyrifera, (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyldimethyl Taurate, Sodium Polymethacrylate, Sodium Polystyrene Sulfonate Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA-Alginate, TEA-Carbomer, Triticum Vulgare (Wheat) Starch, Tromethamine Acrylates/Acrylonitrogens Copolymer, Tromethamine Magnesium Aluminum Silicate, Welan Gum, Yeast Beta-Glucan, Yeast Polysaccharides, and Zea Mays (Corn) Starch.

A first group of particularly preferred thickeners a4) contain at least one structural unit selected from at least one structural unit of formula (I) or its salt forms, or at least one structural unit (II) or its salt forms,

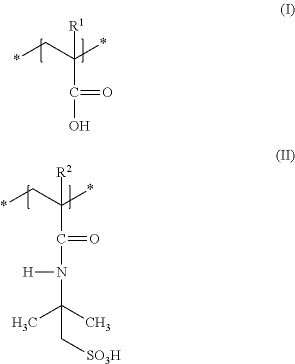

in which R¹ and R⁷ are, independently of one another, a hydrogen atom or a methyl group.

According to the above formulas and all of the following formulas, a chemical bond which is marked with the symbol * stands for a free valence of the corresponding structural fragment.

Particularly preferred anionic polymers having a thickening effect contain at least one structural unit of formula (I). Acrylic acid homopolymers form a first group of particularly preferred thickeners a4).

Particularly preferred thickeners are
polyacrylic acids with the INCI name Carbomer, as are sold for example by the company 3V Sigma under the trade name Synthalen® K or by the company Lubrizol under the trade name Carbopol.

Further particularly preferred anionic polymers having a thickening effect contain at least one structural unit of formula (II). Sulfonic acid copolymers form a second group of particularly preferred thickeners a4). In particular, the copolymers of sulfonic acid-containing monomers with acrylic acid are preferred.

Particularly preferred thickeners are also
sulfonic acid copolymers with the INCI name Sodium Acrylate/Sodium Acryloyldimethyl Taurate Copolymer, as are sold for example by the company SEPPIC under the trade name Simulgel® EC;
sulfonic acid copolymers with the INCI name Sodium Acrylate/Acryloyldimethyl Taurate/Dimethylacrylamide Crosspolymer, as are marketed, for example, by the company SEPPIC under the trade name Simulgel® SMS 88.

A second particularly preferred group of thickeners a4) are the polymeric, anionic, amphiphilic thickeners. Appropriate thickeners preferably include at least one structural unit of formula (III) and at least one structural unit of formula (IV),

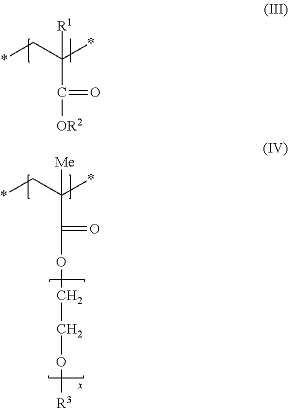

in which
R¹ is a hydrogen atom or a methyl group,
R² is a hydrogen atom or a (C₁ to C₆) alkyl group
R³ is a (C₈ to C₃₀) alkyl group,
M¹ is a physiologically acceptable cation, and
x stands for a whole number from 0 to 35.

Preference is given here in particular to the thickeners with the INCI names Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylates Copolymer, Acrylates/Palmeth-25 Itaconate Copolymer and Acrylates/Steareth-50 Acrylate Copolymer.

Particularly preferred thickeners are
thickeners with the INCI name Acrylates/Steareth-20 Methacrylate Copolymer, as are sold, for example, under the trade name Aculyn® 22 by the company Rohm&Haas;
thickeners with the INCI name Acrylates/Steareth-20 Methacrylate Crosspolymer, as are sold, for example, under the trade name Aculyn® 88 by the company Rohm&Haas;
thickeners with the INCI name Acrylates/Steareth-20 Itaconate Copolymer, as are sold, for example, under the trade name Structure 2001 by the company National Starch.

Other polymeric, anionic, amphiphilic thickeners are characterised by long-chain alkyl substituents. This group includes, for example, the compounds with the INCI names Acrylates/Stearyl Methacrylate Copolymer, Acrylates/Vinyl Isodecanoate Crosspolymer.

A final group of particularly preferred thickeners are lastly
thickeners with the INCI name Acrylates/C₁₀₋₃₀ Alkyl Acrylate Crosspolymer, as are sold, for example, under the trade name Carbopol Ultrez 21 by the company Lubrizol.

A third example of a preferred further active ingredient and auxiliary are the emulsifiers a5). In terms of manufacturability, ease of application and cosmetic effect of cosmetic compositions as contemplated herein it has proven to be advantageous if the proportion by weight of the emulsifier a5) in the total weight of the cosmetic preparation a) is from about 0.02 to about 4.0 wt %, preferably about 0.05 to about 2.0 wt % and in particular about 0.1 to about 1 wt %.

A first group of preferred emulsifiers are the poly (C2-C3) alkylene glycol-modified silicones a5). Particularly preferred are poly (C₂-C₃) alkylene glycol-modified silicones from the group of alkoxylated dimethicones, in particular the
ethoxylated dimethicones with the INCA name PEG-x Dimethicone with x=2 to 20, preferably 3 to 17 and in particular 11 or 12;
the ethoxylated dimethicones with the INCI name Bis-PEG-y Dimethicone with x=3 to 25, preferably 4 to 20;
the ethoxylated/propoxylated dimethicone with the INCI name PEG/PPG a/b Dimethicone, wherein a and b are, independently of one another, numbers from 2 to 30, preferably from 12 to 24 and in particular from 14 to 20;
the ethoxylated/propoxylated dimethicone with the INCI name bis-PEG/PPG-c/d Dimethicone, wherein c and d are, independently of one another, numbers from 10 to 25, preferably 14 to 20, and in particular 14 to 16;
the ethoxylated/propoxylated dimethicone with the INCI name bis-PEG/PPG-e/f PEG/PPG Dimethicone, wherein e, f, g and h, independently of one another, are numbers from 10 to 20, preferably 14 to 18, and in particular are 16.

The use of poly (C2-C3) alkylene glycol-modified silicone a: e group of ethoxylated/propoxylated dimethicones is particularly preferred.

A second group of preferred emulsifiers are the ethoxylated fatty alcohols. An ethoxylated fatty alcohol as contemplated herein is understood to mean an addition product of ethylene oxide with a fatty alcohol, wherein the degree of ethoxylation specifies the molar amount of ethylene oxide (EO) that was deposited on average per mol of fatty alcohol. Polyethoxylated fatty alcohols according to the present disclosure are preferably chosen from polyethoxylated, linear or branched, saturated or unsaturated fatty alcohols, preferably having a chain length of 8 to 22 carbon atoms.

Depending on the production method, the ethoxylated fatty alcohols as contemplated herein arise as a mixture with a different degree of ethoxylation distribution. As contemplated herein, these surfactants are therefore characterised according to the average degree of ethoxylation. This is usually identifiable as the number after the fatty alcohol suffix “eth-” in the INCI name. The agents as contemplated herein preferably contain polyethoxylated fatty alcohols having a degree of ethoxylation of 2 to 29, preferably having a degree of ethoxylation of 2 to 25. more preferably of 3 to 20.

Preferred ethoxy laced fatty alcohols having an average degree of ethoxylation of 2 to 29 are, for example, Laureth-2, Oleth-2, Ceteareth-2, Laneth-2, Laureth-3, Oleth-3, Ceteareth-3, Laureth-4, Oleth-4, Ceteareth-4, Laneth-4, Laureth-5, Oleth-5, Ceteareth-5, Laneth-5, Deceth-7, Laureth-7, Oleth-7, Coceth-7, Ceteth-7, Ceteareth-7, C11-15 Pareth-7, Laureth-9, Oleth-9, Ceteareth-9, Laureth-10, Oleth-10, Beheneth-10, Ceteareth-10, Laureth-12, Ceteareth-12, Trideceth-12, Ceteth-15, Laneth-15, Ceteareth-15, Laneth-16, Ceteth-16, Oleth-16, Steareth-16, Oleth-20, Ceteth-20, Ceteareth-20, Laneth-20, Steareth-21, Ceteareth-23, Ceteareth-25, Ceteareth-27. The use of a mixture of Steareth-2 and Steareth-21 is particularly preferred.

Additional nourishing substances can be cited in particular as further suitable active ingredients or auxiliaries. For example, the agent can contain, as nourishing substance, at least one protein hydrolysate and/or one of its derivatives. Protein hydrolysates are product mixtures obtained by acid-, base- or enzyme-catalysed degradation of proteins (albumins). The term protein hydrolysates is also understood as contemplated herein to mean total hydrolysates as well as individual amino acids and their derivatives and mixtures of different amino acids. The molecular weight of the protein hydrolysates usable as contemplated herein is between about 75, the molecular weight for glycine, and about 200,000, and preferably the molecular weight is about 75 to about 50,000, and very particularly preferably about 75 to about 20,000 Daltons.

As a nourishing substance, the agent as contemplated herein may further comprise at least one vitamin, one provitamin, one vitamin precursor and/or one of the derivatives thereof. Here, as contemplated herein, preferred vitamins, provitamins and vitamin precursors are those that are commonly assigned to the groups A, B, C, E, F and H.

Further nourishing substances are panthenol, caffeine, nicotinamide, and sorbitol.

As a nourishing substance, the agents as contemplated herein may further comprise at least one plant extract, but also mono- or oligosaccharides and/or lipids.

The composition of some particularly preferred cosmetic preparations as contemplated herein can be taken from the following tables (values in wt % based on the total weight of the cosmetic agent unless otherwise specified). That said above with regard to the cosmetic preparations a) as contemplated herein applies mutatis mutandis with respect to further preferred embodiments of these particularly preferred compositions.

| | Formula 1 | Formula 2 | Formula 3 | Formula 4 | Formula 5 |
|---|---|---|---|---|---|
| polar solvent | 45 to 95 | 60 to 92 | 70 to 90 | 88 | 82 |
| a1) | | | | | |
| fatty | 1.0 to 25 | 2.0 to 22 | 4.0 to 20 | 6.3 | 12.4 |
| substance | | | | | |
| a2) | | | | | |
| optional | to 100 | to 100 | to 100 | to 100 | to 100 |
| additives | | | | | |

| | Formula 6 | Formula 7 | Formula 8 | Formula 9 | Formula 10 |
|---|---|---|---|---|---|
| water | 45 to 95 | 60 to 92 | 70 to 90 | 88 | 82 |
| fatty | 1.0 to 25 | 2.0 to 22 | 4.0 to 20 | 6.3 | 12.4 |
| substance | | | | | |
| a2) | | | | | |
| optional | to 100 | to 100 | to 100 | to 100 | to 100 |
| additives | | | | | |

| | Formula | Formula | Formula | Formula | |
|---|---|---|---|---|---|
| | 11 | 12 | 13 | 14 | Formula 15 |
| polar | 45 to 95 | 60 to 92 | 70 to 90 | 88 | 82 |
| solvent a1) | | | | | |
| wax a2 | 1.0 to 25 | 2.0 to 22 | 4.0 to 20 | 6.3 | 12.4 |
| optional | to 100 | to 100 | to 100 | to 100 | to 100 |
| additives | | | | | |

| | Formula | Formula | Formula | Formula | |
|---|---|---|---|---|---|
| | 16 | 17 | 18 | 19 | Formula 20 |
| polar | 45 to 95 | 60 to 92 | 70 to 90 | 88 | 82 |
| solvent a1) | | | | | |
| oil a2 | 1.0 to 25 | 2.0 to 22 | 4.0 to 20 | 6.3 | 12.4 |
| optional | to 100 | to 100 | to 100 | to 100 | to 100 |
| additives | | | | | |

| | Formula | Formula | Formula | Formula | Formula |
|---|---|---|---|---|---|
| | 21 | 22 | 23 | 24 | 25 |
| polar | 45 to 95 | 60 to 92 | 70 to 90 | 88 | 82 |
| solvent a1) | | | | | |
| fatty | 1.0 to 25 | 2.0 to 22 | 4.0 to 20 | 6.3 | 12.4 |
| substance | | | | | |
| a2) | | | | | |
| film-forming | 0.05 to 20 | 0.05 to 15 | 0.1 to 10 | 2.5 | 4.2 |
| polymer a3) | | | | | |
| optional | to 100 | to 100 | to 100 | to 100 | to 100 |
| additives | | | | | |

| | Formula | Formula | Formula | Formula | Formula |
|---|---|---|---|---|---|
| | 26 | 27 | 28 | 29 | 30 |
| water | 45 to 95 | 60 to 92 | 70 to 90 | 88 | 82 |
| fatty | 1.0 to 25 | 2.0 to 22 | 4.0 to 20 | 6.3 | 12.4 |
| substance | | | | | |
| a2) | | | | | |
| film-forming | 0.05 to 20 | 0.05 to 15 | 0.1 to 10 | 2.5 | 4.2 |
| polymer a3) | | | | | |
| optional | to 100 | to 100 | to 100 | to 100 | to 100 |
| additives | | | | | |

| | Formula | Formula | Formula | Formula | Formula |
|---|---|---|---|---|---|
| | 31 | 32 | 33 | 34 | 35 |
| polar | 45 to 95 | 60 to 92 | 70 to 90 | 88 | 82 |
| solvent a1) | | | | | |
| wax a2) | 1.0 to 25 | 2.0 to 22 | 4.0 to 20 | 6.3 | 12.4 |
| film-forming | 0.05 to 20 | 0.05 to 15 | 0.1 to 10 | 2.5 | 4.2 |
| polymer a3) | | | | | |
| optional | to 100 | to 100 | to 100 | to 100 | to 100 |
| additives | | | | | |

| | Formula | Formula | Formula | Formula | Formula |
|---|---|---|---|---|---|
| | 36 | 37 | 38 | 39 | 40 |
| polar | 45 to 95 | 60 to 92 | 70 to 90 | 88 | 82 |
| solvent a1) | | | | | |
| oil a2) | 1.0 to 25 | 2.0 to 22 | 4.0 to 20 | 6.3 | 12.4 |
| film-forming | 0.05 to 20 | 0.05 to 15 | 0.1 to 10 | 2.5 | 4.2 |
| polymer a3) | | | | | |
| optional | to 100 | to 100 | to 100 | to 100 | to 100 |
| additives | | | | | |

| | Formula | Formula | Formula | Formula | Formula |
|---|---|---|---|---|---|
| | 41 | 42 | 43 | 44 | 45 |
| polar | 45 to 95 | 60 to 92 | 70 to 90 | 88 | 82 |
| solvent a1) | | | | | |
| fatty | 1.0 to 25 | 2.0 to 22 | 4.0 to 20 | 6.3 | 12.4 |
| substance | | | | | |
| a2) | | | | | |
| thickener | 0.05 to 10 | 0.05 to 7.0 | 0.1 to 5.0 | 1.4 | 3.2 |
| a4) | | | | | |
| optional | to 100 | to 100 | to 100 | to 100 | to 100 |
| additives | | | | | |

| | Formula | Formula | Formula | Formula | Formula |
|---|---|---|---|---|---|
| | 46 | 47 | 48 | 49 | 50 |
| water | 45 to 95 | 60 to 92 | 70 to 90 | 88 | 82 |
| fatty | 1.0 to 25 | 2.0 to 22 | 4.0 to 20 | 6.3 | 12.4 |
| substance | | | | | |
| a2) | | | | | |
| thickener | 0.05 to 10 | 0.05 to 7.0 | 0.1 to 5.0 | 1.4 | 3.2 |
| a4) | | | | | |
| optional | to 100 | to 100 | to 100 | to 100 | to 100 |
| additives | | | | | |

| | Formula | Formula | Formula | Formula | Formula |
|---|---|---|---|---|---|
| | 51 | 52 | 53 | 54 | 55 |
| polar | 45 to 95 | 60 to 92 | 70 to 90 | 88 | 82 |
| solvent a1) | | | | | |
| wax a2) | 1.0 to 25 | 2.0 to 22 | 4.0 to 20 | 6.3 | 12.4 |
| thickener a4) | 0.05 to 10 | 0.05 to 7.0 | 0.1 to 5.0 | 1.4 | 3.2 |
| optional | to 100 | to 100 | to 100 | to 100 | to 100 |
| additives | | | | | |

| | Formula | Formula | Formula | Formula | Formula |
|---|---|---|---|---|---|
| | 56 | 57 | 58 | 59 | 60 |
| polar | 45 to 95 | 60 to 92 | 70 to 90 | 88 | 82 |
| solvent a1) | | | | | |
| oil a2) | 1.0 to 25 | 2.0 to 22 | 4.0 to 20 | 6.3 | 12.4 |
| thickener a4) | 0.05 to 10 | 0.05 to 7.0 | 0.1 to 5.0 | 1.4 | 3.2 |
| optional | to 100 | to 100 | to 100 | to 100 | to 100 |
| additives | | | | | |

| | Formula | Formula | Formula | Formula | Formula |
|---|---|---|---|---|---|
| | 61 | 62 | 63 | 64 | 65 |
| polar | 45 to 95 | 60 to 92 | 70 to 90 | 88 | 82 |
| solvent a1) | | | | | |
| fatty | 1.0 to 25 | 2.0 to 22 | 4.0 to 20 | 6.3 | 12.4 |
| substance | | | | | |
| a2) | | | | | |
| emulsifier | 0.02 to 4.0 | 0.05 to 2.0 | 0.1 to 1.0 | 0.2 | 0.4 |
| a5) | | | | | |
| optional | to 100 | to 100 | to 100 | to 100 | to 100 |
| additives | | | | | |

| | Formula | Formula | Formula | Formula | Formula |
|---|---|---|---|---|---|
| | 66 | 67 | 68 | 69 | 70 |
| water | 45 to 95 | 60 to 97 | 70 to 90 | 88 | 82 |
| fatty | 1.0 to 25 | 2.0 to 22 | 4.0 to 20 | 6.3 | 12.4 |
| substance | | | | | |
| a2) | | | | | |
| emulsifier | 0.02 to 4.0 | 0.05 to 20. | 0.1 to 1.0 | 0.2 | 0.4 |
| a5) | | | | | |
| optional | to 100 | to 100 | to 100 | to 100 | to 100 |
| additives | | | | | |

| | Formula | Formula | Formula | Formula | Formula |
|---|---|---|---|---|---|
| | 71 | 72 | 73 | 74 | 75 |
| polar | 45 to 95 | 60 to 92 | 70 to 90 | 88 | 82 |
| solvent a1) | | | | | |
| wax a2) | 1.0 to 25 | 2.0 to 22 | 4.0 to 20 | 6.3 | 12.4 |
| emulsifier | 0.02 to 4.0 | 0.05 to 2.0 | 0.1 to 1.0 | 0.2 | 0.4 |
| a5) | | | | | |
| optional | to 100 | to 100 | to 100 | to 100 | to 100 |
| additives | | | | | |

| | Formula | Formula | Formula | Formula | Formula |
|---|---|---|---|---|---|
| | 76 | 77 | 78 | 79 | 80 |
| polar | 45 to 95 | 60 to 92 | 70 to 90 | 88 | 82 |
| solvent a1) | | | | | |
| oil a2) | 1.0 to 25 | 2.0 to 22 | 4.0 to 20 | 6.3 | 12.4 |
| emulsifier | 0.02 to 4.0 | 0.05 to 2.0 | 0.1 to 1.0 | 2.5 | 4.2 |
| a5) | | | | | |
| optional | to 100 | to 100 | to 100 | to 100 | to 100 |
| additives | | | | | |

| | Formula | Formula | Formula | Formula | Formula |
|---|---|---|---|---|---|
| | 81 | 82 | 83 | 84 | 85 |
| water | 45 to 95 | 60 to 92 | 70 to 90 | 88 | 82 |
| fatty | 1.0 to 25 | 2.0 to 22 | 4.0 to 20 | 2.3 | 8.4 |
| substance | | | | | |
| a2) | | | | | |
| film-forming | 0.05 to 20 | 0.05 to 15 | 0.1 to 10 | 2.5 | 4.2 |
| polymer a3) | | | | | |
| thickener a4) | 0.05 to 10 | 0.05 to 7.0 | 0.1 to 5.0 | 1.4 | 3.2 |
| emulsifier | 0.02 to 4.0 | 0.05 to 2.0 | 0.1 to 1.0 | 0.2 | 0.4 |
| a5) | | | | | |
| optional | to 100 | to 100 | to 100 | to 100 | to 100 |
| additives | | | | | |

| | Formula | Formula | Formula | Formula | Formula |
|---|---|---|---|---|---|
| | 86 | 87 | 88 | 89 | 90 |
| water | 45 to 95 | 60 to 92 | 70 to 90 | 88 | 82 |
| wax a2) | 1.0 to 25 | 2.0 to 22 | 4.0 to 20 | 2.3 | 8.4 |
| vinyl- | 0.05 to 20 | 0.05 to 15 | 0.1 to 10 | 2.5 | 4.2 |
| pyrrolidone | | | | | |
| copolymer a3) | | | | | |
| thickener a4) | 0.05 to 10 | 0.05 to 7.0 | 0.1 to 5.0 | 1.4 | 3.2 |
| emulsifier a5) | 0.02 to 4.0 | 0.05 to 2.0 | 0.1 to 1.0 | 0.2 | 0.4 |
| optional | to 100 | to 100 | to 100 | to 100 | to 100 |
| additives | | | | | |

| | Formula | Formula | Formula | Formula | Formula |
|---|---|---|---|---|---|
| | 91 | 92 | 93 | 94 | 95 |
| water | 45 to 95 | 60 to 92 | 70 to 90 | 88 | 82 |
| oil a2) | 1.0 to 25 | 2.0 to 22 | 4.0 to 20 | 2.3 | 8.4 |
| film-forming | 0.05 to 20 | 0.05 to 15 | 0.1 to 10 | 2.5 | 4.2 |
| polymer a3) | | | | | |
| sulfonic acid | 0.05 to 10 | 0.05 to 7.0 | 0.1 to 5.0 | 1.4 | 3.2 |
| copolymer a4) | | | | | |
| emulsifier a5) | 0.02 to 4.0 | 0.05 to 2.0 | 0.1 to 1.0 | 0.2 | 0.4 |
| optional | to 100 | to 100 | to 100 | to 100 | to 100 |
| additives | | | | | |

Very particularly preferred cosmetic preparations contain, besides the above-described constituents a1) to a5), only small amounts of other active ingredients and auxiliaries. Cosmetic preparations characterised in that the proportion by weight of the components a1), a2) and, if present, a3), a4) and/or a5) in the total weight of the cosmetic preparation is at least about 80 wt %, preferably at least about 87 wt %, particularly preferably at least about 92 wt % are particularly preferred because of their simple manufacture and good cosmetic effect. Very particularly preferred cosmetic preparations consist, based on their total weight, to an extent of at least about 80 wt %, preferably at least about 85 wt % and in particular at least about 87 wt % of the constituents a1), a2) and a3).

As stated at the outset, cosmetic preparations as contemplated herein a) are particularly suitable for application utilizing a device for flash evaporation. Another subject of the present application is therefore the use of a cosmetic preparation a) containing, based on its total weight,
a1) about 45 to about 95 wt % of at least one polar solvent;
a2) about 1.0 to about 25 wt % orf at least one fatty substance;
   as a process material in a device for flash evaporation.

An additional subject of the present application is the use of a product as contemplated herein for acting on keratin-containing fibres, in particular human hair, utilizing a cosmetic preparation a) or for the temporary shaping of keratin-containing fibres, particularly human hair.

A further subject of the present application is a method for the temporary shaping of keratin-containing fibres, particularly human hair, in which the keratin-containing fibres are acted on utilizing a device for flash evaporation with a cosmetic preparation a) containing, based on its total weight
a1) about 45 to about 95 wt % of at least one polar solvent;
a2) about 1.0 to about 25 wt % of at least one fatty substance.

The cosmetic preparation a) is preferably converted utilizing the device for flash evaporation into a spray which subsequently acts on the keratin-containing fibres.

In order to achieve a sufficient spray effect, the cosmetic preparation is preferably heated to temperatures above the boiling point of the polar solvent or solvent mixture contained in the cosmetic preparation a).

If the polar solvent is water or solvent mixtures with a water content above about 50 wt % (based on the total weight of the solvent mixture), the cosmetic preparation is preferably heated to temperatures above about 100° C., preferably to temperatures of about 100° C. and about 240° C., particularly preferably to temperatures of from about: 140° C. to about 160° C.

The overpressure achieved by the heating of the cosmetic preparation a), in the cases where the polar solvent is water or solvent mixtures with a water content above about 50 wt % (based on the total weight of the solvent mixture , is preferably between abot about 8 bar, preferably between about 1.2 and about 4 bar.

A preferred subject of the application is a method for changing the colour of keratin-containing fibres, particularly human hair, in which the keratin-containing fibres are acted on utilizing a device for flash evaporation with a cosmetic preparation a) containing, based on its total weight
a1) about 45 to about 95 wt % of at least one polar solvent;
a2) about 1.0 to about 25 wt % of at least one fatty substance;
   wherein
a portion of the cosmetic preparation a) disposed in a storage tank is transferred from this storage tank, inside which there is a pressure corresponding to the ambient pressure, into a container b1);
the access between storage tank and container b1) is then interrupted by a component for flow control, which the flow of the cosmetic preparation a) from the storage tank into the container b1) can be interrupted;
subsequently, the cosmetic preparation a) disposed in the container b1) closed off with respect to the surrounding environment is heated by a heater, so that the pressure inside the container b1) rises to values above ambient pressure, preferably to values between about 1.1 and about 8 bar, in particular to values between about 1.2 and 4 about bar;
subsequently, the container b1), which is at a pressure above ambient pressure, is opened in a manner such that the outlet of at least a portion, preferably at least about 50 wt %, preferably at least about 80 wt %, and in particular at least about 90 wt % of the cosmetic preparation disposed in the container b1) from the container b1) into the surrounding environment is pressure-relieved, with a reduction of the pressure prevailing in the container b1) at the time of the container opening.

The pressure of the cosmetic preparation is reduced preferably such that a spray mist of the cosmetic preparation a) is formed.

The cosmetic preparation a) relieved of pressure from the container b1) is preferably applied to keratin fibres, particularly human hair.

Methods during the course of which the cosmetic preparation pressure-relieved from the container b1) is passed through a nozzle prior to acting on the keratin fibres are particularly preferred.

That said with regard to the cosmetic preparations a) as contemplated herein and to the device for flash evaporation h) applies mutatis mutandis with respect to further preferred embodiments of the uses as contemplated herein and of the method as contemplated herein.

The agents, uses and methods as contemplated herein and some of the preferred embodiments thereof are summarised by the following points:
1. A cosmetic product, comprising
   a) a cosmetic preparation, containing, based on its total weight,
      a1) about 45 to about 95 wt % of at least one polar solvent;
      a2) about 1.0 to about 25 wt % of at least one fatty substance;
   b) a device for flash evaporation of the cosmetic preparation a)
2. The cosmetic product according to point 1, characterised in that the device for flash evaporation comprises a container b1) and a heater h2) and is designed such that
   the cosmetic preparation a) can he received in the interior of the container b1),
   the interior of the container b1) filled at least in part with the cosmetic preparation a) can be closed,
   the cosmetic preparation a) may be healed in the closed interior of the container b1) utilizing the heater b2) with a resultant increase in pressure,
   the heated cosmetic preparation a) can be pressure-relieved into the surrounding environment from the interior of the container b1) with a reduction in pressure.
3. The cosmetic product according to any one of the preceding points, characterised in that the proportion by weight of the polar solvent a1) in the total weight of the cosmetic preparation a) is about 60 to about 92 wt %, preferably about 70 to about 90 wt %.
4. The cosmetic product according to any one of the preceding points, characterised in that the polar solvent a1) has a boiling point (20° C., 1013 mbar) between about 50 and about 110° C., preferably between about 70 and about 105° C.
5. The cosmetic products according to any one of the preceding points, characterised in that the polar solvent a1) is selected from the croup ethanol, isopropanol, glycerol and water.
6. The cosmetic products according to any one of the preceding points, characterised in that the proportion by weight of water and ethanol in the total weight of the polar solvent a1) is preferably at least about 60 wt %, preferably at least about 70 wt %, particularly preferably at least about 80 wt %, and in particular at least about 90 wt %.
7. The cosmetic products according to any onef the preceding points, characterised in that the proportion by weight of water in the total weight of the polar solvent a1) is more than about 70 wt %, preferably more than about 80 wt %, and in particular more than about 90 wt %.
8. The cosmetic product according to any one of the preceding points, characterised in that the proportion by weight of the fatty substance a2) in the total weight of the cosmetic preparation a) is about 2.0 to about 22 wt %, preferably about 4.0 to about 20 wt %.
9. The cosmetic product according to any one of the preceding points, characterised in that the fatty substance a2) is selected from the group of waxes.
10. The cosmetic product according to any one of the preceding points, characterised in that the fatty substance a2) is selected from the group consisting of beeswax (Cera Alba), carnauba wax and microcrystalline waxes (microcrystalline paraffins)
11. The cosmetic product according to any one of the preceding points, characterised in that the fatty substance a2) is selected from the group of oils.
12. The cosmetic product according to any one of the preceding points, characterised in that the fatty substance a2) is selected from the group of silicone oils, paraffin oils, vegetable oils and ester oils, preferably from the group of silicone oils, paraffin oils and vegetable oils.
13. The cosmetic product according to any one of the preceding points, characterised in that the cosmetic preparation a) contains, based on its total weight, about 0.05 to about 20 wt %, preferably about 005 to about 15 wt %, and in particular about 0.1 to about 10 wt % of a film-forming polymer a3).
14. The cosmetic product according to point 13, characterised in that the film-forming polymer a3) is selected from the group of vinylpyrrolidione homo- and copolymers, preferably from the group of polyvinylpyrrolidones, the copolymers of vinylpyrrolidone and vinyl acetate, the copolymers of vinylpyrrolidone and dimethylaminopropyl methacrylamide and the copolymers of vinylpyrrolidone with vinyl caprolactam and dimethylaminoethyl methacrylate.
15. The cosmetic product according to point 13, characterised in that the film-forming polymer a3) is selected from the group of copolymers of i) N-tert-octylacrylamide, ii) acrylic acid, iii) ten-butylaminoethyl methacrylate, iv) and optionally further monomers.
16. The cosmetic product according to any one of the preceding points, characterised in that the cosmetic preparation a) contains, based on its total weight, about 0.05 to about 10 wt %, preferably about 0.05 to about 7.0 wt %, and in particular about 0.1 to about 5.0 wt % of a thickener a4).
17. The cosmetic product according to any of the preceding points, characterised in that the thickener a4) is selected from the group of acrylic acid homopolymers Carbomer, etc.).
18. The cosmetic product according to any one of the preceding points, characterised in that the thickener a4) is selected from the group of sulfonic acid copolymers (Simulgel, etc.).
19. The cosmetic product according to any of the preceding points, characterised in that the thickener a4) is selected from the group of compounds with the INCI name Carbomer, Sodium Acrylate/Sodium Acryloyldimethyl Taurate Copolymer Sodium Acrylate/Acryloyldimethyl Taurate/Dimethylacrylamide Crosspolymer, Acrylates/Steareth-20 Methacrylate Copolymer, Acrylates/Steareth-20 Methacrylate Crosspolymer, Acrylates/Steareth-20 Itaconate Copolymer, and Acrylates/C₁₀₋₃₀ Alkyl Acrylate Crosspolymer.
20. The cosmetic product according to any one of the preceding points, characterised in that the cosmetic preparation a) contains, based on its total weight, about 0.02 to about 4.0 wt %, preferably about 0.05 to about 2.0 wt %, and in particular about 0.1 to about 1.0 wt % emulsifier a5).
21. The cosmetic product according to point 20, characterised in that the emulsifier a5) is selected from the group of poly (C₂-C₃) alkylene glycol-modified silicones and of ethoxylated fatty alcohols.
22. The cosmetic product according to any one of the preceding points, characterised in that the cosmetic preparation a), based on its total weight, consists to an extent of at least about 80 wt %, preferably at least about 87 wt %, and in particular at least about 92 wt % of the constituents a1), a2), and, if present, a3), a4) and/or a5).
23. The cosmetic product according to any one of the preceding points, characterised in that the cosmetic preparation a), based on its total weight, consists to an extent of at least about 80 wt % preferably at least about 85 wt %, and in particular at least about 87 wt % of the constituents a1), a2) and a3).
74. Use of a cosmetic composition a) containing, based on its total weight,
   a1) about 45 to about 95 wt % of at least one polar solvent;
   a2) about 1.0 to about 25 wt %) of at least one fatty substance;
   as a process material in a device for flash evaporation.
25. Use of a product according to any one of points 1 to 23 for acting on keratin-containing fibres, in particular human hair, with a cosmetic preparation a).
26. Use of a product according to any one of 1 to 23 for temporarily shaping keratin-containing fibres, particularly human hair.
27. A method for temporarily shaping keratin-containing fibres, particularly human hair, in which the keratin-containing fibres are acted on utilizing a device for flash evaporation with a cosmetic preparation a), containing, based on its total weight,
   a1) about 45 to about 95 wt % of at least one polar solvent;
   a2) about 1.0 to about 25 wt % of at least one fatty substance.
28. The method according to point 27, characterised in that
   a portion of the cosmetic preparation a) disposed in a storage tank is transferred from this storage tank, inside which there is a pressure corresponding to the ambient pressure, into a container b1);
   the access between storage tank and contain b1) is then interrupted by a component for flow control, which the flow of the cosmetic preparation a) from the storage tank into the container b1) can be interrupted;
   subsequently, the cosmetic preparation a) disposed in the container b1) closed off with respect to the surrounding environment is heated by a heater, so that the pressure inside the container b1) rises to values above ambient pressure, preferably to values between about 1.1 and about 8 bar, in particular to values between about 1.2 and about 4 bar;
   subsequently, the container 1), which is at a pressure above ambient pressure, is opened in a manner such that the outlet of at least a portion, preferably at least about 50 wt %, preferably at least about 80 wt %, and in particular at least about 90 wt % of the cosmetic preparation disposed in the container b1) from the container b1) into the surrounding environment is pressure-relieved, with a reduction of the pressure prevailing in the container b1) at the time of the container opening.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product, comprising:
a cosmetic preparation, comprising, based on its total weight,
about 45 to about 95 wt % of at least one polar solvent, and
about 1.0 to about 25 wt % of at least one fatty substance; and
a device for flash evaporation of the cosmetic preparation a).

2. The cosmetic product according to claim 1, wherein the device for flash evaporation comprises a container and a heater and is designed such that
the cosmetic preparation can be received in the interior of the container,
the interior of the container filled at least in part with the cosmetic preparation can be closed,
the cosmetic preparation may be heated in the closed interior of the container by the heater with a resultant increase in pressure,
the heated cosmetic preparation can be pressure-relieved into the surrounding environment from the interior of the container with a reduction in pressure.

3. The cosmetic product according to claim 1, wherein the proportion by weight of the polar solvent in the total weight of the cosmetic preparation is about 60 to about 92 wt %.

4. The cosmetic product according to claim 1, wherein the polar solvent is selected from the group of ethanol, isopropanol, glycerol, water, and combinations thereof.

5. The cosmetic product according to claim 1, wherein the proportion by weight of the fatty substance in the total weight of the cosmetic preparation is about 2.0 to about 22 wt %.

6. The cosmetic product according to claim 1, wherein the cosmetic preparation comprises, based on its total weight, about 0.05 to about 20 wt % of a film-forming polymer.

7. The cosmetic product according to claim 1, wherein the cosmetic preparation comprises, based on its total weight, about 0.05 to about 10 wt % of a thickener.

8. A cosmetic composition comprising, based on its total weight,
about 45 to about 95 wt % of at least one polar solvent, and
about 1.0 to about 25 wt % of at least one fatty substance; and
wherein the cosmetic composition is utilized as a process material in a device for flash evaporation.

9. The cosmetic product according to claim 1 utilized for acting on keratin-containing fibres with a cosmetic preparation a).

10. The cosmetic product according to claim 1 utilized for temporarily shaping keratin-containing fibres.

11. A method for temporarily shaping keratin-containing fibres in which the keratin-containing fibres are acted on by a device for flash evaporation with a cosmetic preparation comprising, based on its total weight,
about 45 to about 95 wt % of at least one polar solvent; and
about 1.0 to about 25 wt % of at least one fatty substance.

12. The method according to claim 11, comprising:
transferring a portion of the cosmetic preparation disposed in a storage tank from this storage tank, inside which there is a pressure corresponding to the ambient pressure, into a container;
interrupting the access between storage tank and the container by a component for flow control to interrupt the flow of the cosmetic preparation from the storage tank into the container;
heating the cosmetic preparation disposed in the container closed off with respect to the surrounding environment by a heater, so that the pressure inside the container rises to values above ambient pressure;
opening the container, which is at a pressure above ambient pressure, such that the outlet of at least a portion of the cosmetic preparation disposed in the container from the container into the surrounding environment is pressure-relieved, with a reduction of the pressure prevailing in the container at the time of the container opening.

13. The cosmetic product according to claim 1, wherein the proportion by weight of the polar solvent in the total weight of the cosmetic preparation is about 70 to about 90 wt. %.

14. The cosmetic product according to claim 1, wherein the proportion by weight of the fatty substance in the total weight of the cosmetic preparation is about 4.0 to about 20 wt %.

15. The cosmetic product according to claim 1, wherein the cosmetic preparation comprises, based on its total weight, about 0.05 to about 15 wt % of a film-forming polymer.

16. The cosmetic product according to claim 1, wherein the cosmetic preparation comprises, based on its total weight, about 0.1 to about 10 wt % of a film-forming polymer.

17. The cosmetic product according to claim 1, wherein the cosmetic preparation comprises, based on its total weight, about 0.05 to about 7.0 wt % of a thickener.

18. The cosmetic product according to claim 1, wherein the cosmetic preparation comprises, based on its total weight, about 0.1 to about 5.0 wt % of a thickener.

19. The method according to claim 12, wherein the values above ambient pressure are values between about 1.1 and about 8 bar.

20. The method according to claim 12, wherein the at least a portion of the cosmetic preparation is in an amount of at least about 50 wt. %.
